(19) United States
(12) Patent Application Publication
(10) Pub. No.: US 2021/0371887 A1
(43) Pub. Date: Dec. 2, 2021

(54) METHOD AND APPARATUS FOR THE UTILIZATION OF ZERO FIBER AND OTHER SIDE STREAMS

(71) Applicant: Eino Elias Hakalehto, Kuopio (FI)

(72) Inventor: Eino Elias Hakalehto, Kuopio (FI)

(21) Appl. No.: 17/404,460

(22) Filed: Aug. 17, 2021

Related U.S. Application Data
(63) Continuation-in-part of application No. 15/027,989, filed on Apr. 7, 2016.

(30) Foreign Application Priority Data
Aug. 17, 2020 (FI) 20200056

(51) Int. Cl. C12P 7/56; C12P 7/64; C12P 7/18; C05F 17/20

(52) U.S. Cl. CPC C12P 7/56; C05F 17/20; C12P 7/18; C12P 7/6409

(57) ABSTRACT

A combined process for several biorefinery products obtained from an Undefined Mixed Culture (UMC) type of reaction it is possible to obtain biochemicals, energy gases, soil improvement etc. from a Multipurpose Biorefinery Unit MPBU). The economically beneficial as well as environmentally sustainable results of the arrangement are demonstrated by the integrated process using two reactor systems with zero fiber for the production of lactate in both the reactors pools 1 and 2. Additionally, mannitol can be produced in one of the reactor pools (number 2). It is possible to: a. combine the processes taking into account their biochemical characteristics, b. produce gaseous substances for energy and industrial use, c. obtain organic fertilizers which can be microbiologically upgraded, and d. improve the adjustability for optimization of the various partial reactivities. The chemical production occurs in two pools which advantageously are inoculated simultaneously.

### CROSS-REFERENCE TO RELATED APPLICATION

This application is a continuation-in-part under 35 U.S.C. § 120 based upon co-pending U.S. patent application Ser. No. 15/027,989 filed on Apr. 7, 2016, which is incorporated herein by reference in its entirety.

The present application claims priority under 35 U.S.C. 119(a) to Finland (FI) patent application number 20200056 filed on Aug. 17, 2020, which is incorporated herein by reference in its entirety.

### BACKGROUND

### Technical Field

The present technology relates to a method and apparatus for the utilization of zero fiber and other side streams for use in connection with synchronizing the biochemical and microbiological process utilizing a two reactor pool bioprocessing plant for the creating of mannitol and lactate.

### Background Description

Mannitol that forms as a result of bacterial metabolism normally comes from fructose. In nature, fructose is found, e.g. in hemicellulose (for example in wood material), and in various parts of plants, such as in fruits. Wastes and byproducts that contain fructose are thus well suitable as raw materials for mannitol production.

Fructose can also be formed from raw materials that contain glucose (cellulose, starch, etc.). For example, the starch from corn, barley, potato, or some other plant, can be split, with the help of amylase and amyloglucosidase enzymes, into glucose, which is then, with the help of glucose-isomerase enzymes, into fructose. This is the process used in the production of High Fructose Corn Syrup. It has been developed into an industrially viable product and High Fructose Corn Syrup is an important sweetener. Correspondingly, different sugar industry fractions can be enzymatically processed and their fructose content and respectively their sweetening value can thus be increased.

The broad use of fructose in foodstuffs has lately been hindered by the uncertainty of its healthiness as a component and a sweetener in human nutrition. Fructose breaks down in the body only in the liver and its excessive use has been shown to cause obesity.

Stomach manure is the content of the stomach that forms into the rumen in bovine and other ruminants. In slaughter houses the amount of stomach manure is approximately 50 kg per one bovine. Thus in a slaughterhouse that slaughters 200 animals a day, about 10 tons of stomach manure is gathered per day. Stomach manure is formed naturally also from the slaughtering of other than bovine animals. The utilization of this waste for example by composting or by producing biogas, requires considerable space and thus expensive investments. Thus the economic benefit and increase of cleaning power gained with a fast biotechnical process is meaningful.

Stomach manure microbes exist also in the manure of other slaughter animals. Stomach manure or manure can be combined with other wastes in different waste treatment processes.

In traditional biotechnical processes using micro-organisms as biocatalysts it is usual to have single species or sometimes two species or strains of microbes performing the desired reaction. It is then believed to become more adjustable and controllable as well as more predictable one. However, such microbiological processes seldom take place in natural habitats. Also, in the man-made processes there are often mixed inocula used as a seed. In such cases the microbes can strive for a balance with each other in a mixed culture. Such examples are the biogas production, composting of organic matter, and other bioprocesses taking place in a more ecosystem-like reaction environment. Such mixed substrate milieu can be found in the so called zero fiber waste which is produced in tens of thousands of tons annually as a side-stream of a big paper or cardboard factory. This fibrous material consists of the cellulosic molecules which are too short for e.g. paper-making.

Zero fiber deposit have accumulated in the proximity of many forest industries, often onto the lake or sea bottom or an equivalent reservoir as a sediment (Hakalehto 2018a). Their processing into useful chemical substances has been developed for the production of such organic acids as lactic acid, for instance (Beckinghausen et al. 2019, Hakalehto 2020).

The maturation of any microbial community as a whole occurs in the industrial bioprocess setting in order to obtain the best possible results from one or more biochemical reactions. This also makes the contamination control usually much easier. However, sometimes it is also possible to get several end-products from the same reaction broth. This, in turn, sets up additional complications for the steering of the process and for the adjustment of its parameters. This is particularly true when the conditions have to be changed during the process in order to facilitate first the variable end-product formation of the hydrolysis reactions for obtaining appropriate raw material, and then for the switch to the actual production reaction or for avoiding any extensive end-product inhibition, catabolite repression or other biological regulation mechanism. It is also often advantageous to run a biorefinery process in an oscillating way, where the values for the key parameters change in a cyclic manner (Hakalehto et al. 2008).

The above-mentioned changes or transitions into more complex matrices of control parameters also produce more effective ways for controlling the process conditions provided that the process remains under control. The importance of this increased amount of adjustment tools with more flexibility will be emphasized in the ecosystem-type of bioreactor systems. Also, when the hydrolysis reactions are carried out in the same compartment or simultaneously with the product formation, this requires more sophisticated technologies for the measurement and control. If the production scale is growing, this demand for additional control further increases.

Many microbiological processes in the biorefineries (Hakalehto 2016a,b, 2018a, Den

Boer et al. 2016, Schwede et al. 2017, Beckinghausen et al. 2019) have their counterpart or otherwise corresponding reactions in the digestive or alimentary tract (Hakalehto 2011, 2012, 2013). The concept of BIB (Bacteriological Intestinal Balance) has been developed for describing the internal strive of the digestive microbial ecosystem for establishing a balance. Some features of “habitat dominance by coalition” are discussed previously (Hakalehto 2018b). The basis of the self-control processes by interspecies dominance or succession can be observed between various members of some intestinal strains belonging to the family Enterobacteriaceae in the duodenum (Hakalehto et al. 2008), as well as by the interacting lactobacilli and clostridia in the large intestines (Hakalehto and Hänninen 2012).

The process of so-called Consolidated Bioprocessing (CBP) in practice means a bioprocess simultaneously using both common kinds of biocatalysts, namely the enzymes and the microbial strains. These biocatalysts are used for the processing of the organic polymeric raw materials as biocatalysts.

In fact, the industrial bioprocess broth somewhat resembles the intestinal chyme, both of them being non-aseptic bioreactor systems. This kind of process is advanced by a mixed microbial flora often called as UMC (Undefined Mixed Culture). As like in the alimentary tract, the corresponding process ecosystem is eventually seeking for a balance. Recognizing or identifying such balances as well as the steering or exploiting of them for the improved production of the desired chemicals or gases opens up new opportunities for microbial bioprocesses.

During the more or less fermentative i.e. anoxic processes, multitude of parameters influence the outcome of the reaction. Most of them are adjustable by the operator, which is a significant asset for the optimization of any biological production process. Such processes are characterized by Hakalehto et al. (2016), Hakalehto (2018a) and Jääskeläinen et al. (2016), for instance. The further improvement of these processes is also in the scope of this invention.

The above-mentioned parameters for the microbe process include:
temperature
pH
oxygen content
gassing
viscosity
mixing
pressure
fractionation
gradient formation
   etc.

These adjustable traits essentially influence the outcome of the process. It can be altered also by adding some more strains with desired metabolic or regulatory characteristics into the process.

As it is often beneficial to have various gradients in the biorefinery process in the production broth; the establishment, enforcing and controlling of those gradients gives potential for steering up of the process. The processing goals can be promoted by intelligent adjustment, and also by dividing the process into sequences, phases, different compartments etc. Partial walls or semipermeable membranes can be used for this purpose. Various atmospheres have been created into various parts of the reactor by the above-mentioned means (Hakalehto 2008). According to this previous Patent Application aerobic and anoxic gas mixtures can be led into the different parts of the reactor. This forms gradients which can be beneficial for avoiding the regulatory mechanisms of the various members of the mixed microflora, for instance. Moreover, simultaneous accomplishment of different objectives becomes easier, such as the running of the CBP (Consolidated Bioprocessing) type of reactions (Hakalehto 2015a). This means the integration of enzymatic hydrolysis and the actual microbial process in the one and same reactor.

The common problems in the CBP unit includes different preferential or optional conditions for the enzymatic process and the microbe process. For the former one, the most important core parameters are:
concentration of the enzyme
temperature
pH
enzymatic activity
affinity of the enzyme toward the substrate(s)
duration of the effective time for enzymatic process
self-life of the enzyme molecules
enzyme sensitivity to disturbing factors
regulation of the enzymatic function

The final result of the hydrolysis process is a combination or a selection of these circumstances provided that the major substrate is not the limiting factor. The microbial process and its self-regulation are even more complicated sequences of events and successions of various strains or their different reactivities. Consequently, it is of crucial importance to identify the main reactions, their duration, optimal conditions and drivers, in order to integrate various processes (Hakalehto and Jääskeläinen 2017). Moreover, the use of mixed microflora in the bioprocess may further complicate the control, particularly in the case of multiple products whose manufacturing needs to be optimized simultaneously. Sometimes it can be beneficial to partially separate the various processes or their phases. In any case, various measurements and ways for monitoring the process and its phases are required.

For both the enzymatic process and the subsequent microbial process, the shape of the reactor as well as the reachability of the substrate by the biocatalyst are also important technical parameters when designing the reactor hardware for the CBP. The picture becomes even more complicated one, if several products are produced at the same time. Again we meet the limitations caused by the differing or conflicting requirements of the bioprocess within the one and same reactor system. This variation may occur regardless of the composition of the raw material, whether it is in a solution, broth, suspension, emulsion or on a solid phase. Likewise, the supposed movement or lack of movement, or the stability or instability or lability of the raw materials at any given time point does not eliminate the issue of finding difficulties in getting conditions right for the simultaneous catalytic processes of the successful bioreaction leading to useful results from chemical conversion of microbial metabolites into precious products, those metabolites being obtained from enzymatically degraded macromolecules.

While the above-described devices fulfill their respective, particular objectives and requirements, the aforementioned devices or systems do not describe a method and apparatus for the utilization of zero fiber and other side streams that allows synchronizing the biochemical and microbiological process utilizing a two reactor pool bioprocessing plant for the creating of mannitol and lactate.

A need exists for a new and novel method and apparatus for the utilization of zero fiber and other side streams that can be used for synchronizing the biochemical and microbiological process utilizing a two reactor pool bioprocessing plant for the creating of mannitol and lactate. In this regard, the present technology substantially fulfills this need. In this respect, the method and apparatus for the utilization of zero fiber and other side streams according to the present technology substantially departs from the conventional concepts and designs of the prior art, and in doing so provides an apparatus primarily developed for the purpose of synchronizing the biochemical and microbiological process utilizing a two reactor pool bioprocessing plant for the creating of mannitol and lactate.

### SUMMARY

In view of the foregoing disadvantages inherent in the known types of mannitol production systems and methods, the present technology provides a novel method and apparatus for the utilization of zero fiber and other side streams, and overcomes one or more of the mentioned disadvantages and drawbacks of the prior art. As such, the general purpose of the present technology, which will be described subsequently in greater detail, is to provide a new and novel method and apparatus for the utilization of zero fiber and other side streams and method which has all the advantages of the prior art mentioned heretofore and many novel features that result in a method and apparatus for the utilization of zero fiber and other side streams which is not anticipated, rendered obvious, suggested, or even implied by the prior art, either alone or in any combination thereof.

According to one aspect, the present technology can include a combined process for several biorefinery products obtained from an Undefined Mixed Culture (UMC) type of reaction it is possible to obtain biochemicals, energy gases, soil improvement etc. from a Multipurpose Biorefinery Unit (MPBU). The economically beneficial as well as environmentally sustainable results of the arrangement are demonstrated by the integrated process using two reactor systems with zero fiber for the production of lactate in both the reactors pools 1 and 2. Additionally, mannitol can be produced in one of the reactor pools (number 2). It is possible to

a. combine the processes taking into account their biochemical characteristics,

b. produce gaseous substances for energy and industrial use,

c. obtain organic fertilizers which can be microbiologically upgraded, and

d. improve the adjustability for optimization of the various partial reactivities.

The chemical production occurs in two pools which advantageously are inoculated simultaneously.

The present invention overcomes disadvantages and drawbacks of the prior art. As such, the general purpose of the present invention, which will be described subsequently in greater detail, is to provide a new and improved process and waste treatment for the utilization of intestinal bacteria from slaughtered animals and method which includes many novel features that rare not anticipated, rendered obvious, suggested, or even implied by the prior art, either alone or in any combination thereof.

To attain this, the present invention essentially comprises:

A method characterized in, that mono or disaccharides or their polymers are added into the stomach manure of a slaughtered animal or manure produced by it or refinery sludge, in order to increase the productivity or production levels of wanted biotechnical products.

A method characterized in, that the particular sugar used is sucrose or glucose or fructose or a mixture of them.

A method characterized in, which glucose and fructose can be produced enzymatically as a result of decomposition of starch, cellulose, hemicellulose or other macromolecule.

A method characterized in, that the amount of fructose in the substrate liquid to be added has been added by enzymatic means.

A method characterized in, that the lactic acid bacteria in the stomach manure or naturally in other waste or that are added to it, convert into mannitol the fructose that is in the stomach manure or other waste or forms into it or is added into it.

A method characterized in, that the pH is allowed to lower in stomach manure or other waste as the glucose that is in it, forms into it or is added to it, changes into lactic acid and other acidity increasing substances due to lactic acid bacteria and other microbes performing acetic acid fermentation and other organic acid producing reactions, which simultaneously prevents the decomposition or metabolization of mannitol that has formed into the process liquid.

A method characterized in, that the pH value starts to be raised by adding base when all fructose or most of the fructose has converted into mannitol.

A method characterized in, that the mannitol is removed by crystallization or other methods prior to raising the pH.

A method characterized in, that CO and/or CO₂ containing gas emissions, formed from burning biotechnical fractions or from other combustion reactions, are led into the process liquid to heat it and to facilitate its further exploitation.

A method characterized in, that the lactate formed in the process through fermentation reactions is converted to propionate, i.e. propionic acid.

A method characterized in, that propionic acid can further be converted, for example with the help of Clostridium bacteria, into other organic acids, alcohols or other compounds.

A method characterized in, that into the stomach manure or other waste, such a bacterial strain or combination of different strains are added that help in changing a significant part of lactate into propionate which is collected for burning or for chemical utilization for example as a preservative in feed or food or other products.

A device to use the above-identified method characterized in, that fructose, fructose-containing sugar, byproduct fraction or waste or glucose that is enzymatically converted into fructose, is added into stomach manure, after the adjustment of humidity percentage, in a separate reactor (A) from the bioreaction.

An apparatus characterized in, that after mannitol has been removed the residue of the process liquid is led to a pool, container or reactor (B) in which the pH and temperature are adjusted to be suitable for the production of propionic acid.

An apparatus characterized in, that the microbial inoculums is inoculated from the inoculation fermentor (C) which has a dosing pump for the adjustment of microbial levels.

An apparatus characterized in, that propionic acid can further be converted to other organic acids, alcohols or other compounds.

An apparatus characterized in, that ammonium salts that have formed from stomach manure or other waste as a result of protein decomposition can be precipitated from the process liquid to fertilizers and collected with the process residue or by separation.

According to another aspect, the present technology can include a method for optimizing a simultaneous or interlinked production of lactate or Small Chain Fatty Acids (SCFA's) and mannitol. The method can include the steps of utilizing a first reactor pool for the production of lactate or SCFA's and a second reactor pool for the production of mannitol using rumen bacteria as biocatalysts in such a way that after recovery of the mannitol a residual process fluid is applied to the first reactor pool for further elevating both the lactate or SCFA's and the mannitol levels of a biorefining as a whole.

In some or all embodiments, inoculation of the first and second reactor pools can be carried out simultaneously.

In some or all embodiments, zero fiber or a cellulosic material in the first reactor pool can be used as a main source of glucose in the first reactor pool.

In some or all embodiments, cellulolytic enzymes can be used for hydrolysis of the zero fiber or cellulosic material, at least partially in a Consolidated Bioprocessing (CBP) mode, simultaneously with microbial processes.

In some or all embodiments, fructose containing side streams can be used as a raw material source for the production of the mannitol.

In some or all embodiments, the mannitol can be recovered either from the second reactor pool into which the fructose and the rumen bacteria had been added, or from the first reactor pool if residues of the second reactor pool are transferred to the first reactor pool without prior recovery of the mannitol.

In some or all embodiments, the cellulosic material being the zero fiber, can be used in both of the first reactor pool and the second reactor pool amongst other raw materials.

In some or all embodiments, purification of the lactate or the SCFA's can be carried out of the residues of both of the first reactor pool and the second reactor pool either separately or as combined to each other.

In some or all embodiments, hydrogen in a bubble flow can be collected by suction for further use as an energy gas or a reducing agent.

In some or all embodiments, a final fraction with solid particles or suspension can be collected for soil improvement or organic fertilization purposes.

In some or all embodiments, the final fraction of the biorefinery can be upgraded as soil improvement by using bacteria of the species Clostridium pasteurianum or other autonomously nitrogen-fixing species for increasing a soil nitrogen content available for plant growth.

In some or all embodiments, the final fraction can be used for replacing or increasing a soil humic fraction.

According to another aspect, the present technology can include an apparatus for using any of the above described method, wherein the first reactor pool and the second reactor pool are arranged into such a position with respect to each other that residues of the second reactor pool are added a shortest way into the first reactor pool in a process phase that corresponds to a time point in the first reactor pool and process.

In some or all embodiments, a process fluid, flow, broth or suspension can be moving forwards from a beginning to an end of the process by way of rotors, screws, blows or paddlewheels.

In some or all embodiments, the apparatus can further include sensors or other measurement systems for measuring temperature, pH, turbidity, contents of various gases, conductivity, pO2, pCO2, impedance, viscosity, glucose or fructose content, or any other relevant or measurable parameter for the bioprocess. The sensors can be situated at any point of the process or process flow in any of the first reactor pool and the second reactor pool.

In some or all embodiments, the process can be adjusted in any of the first reactor pool and the second reactor pool with respect to one or more chosen parameters at any time point during the process flow. The parameters can be a temperature of 28-32° C. for a lactate-producing LAB population in the first reactor pool, and at 37-42° C. for a corresponding population in the second reactor pool.

In some or all embodiments, process control and adjustment or addition of reagents or water is being facilitated by results of measurements by the sensors.

In some or all embodiments, the mannitol can be recovered by crystallization or by any other method carried out in a separate container or series of containers from the fluid of the second reactor pool.

In some or all embodiments, the lactate can be a main product in the first reactor pool, whereas the lactate is an additional product in the second reactor pool with same equipment being used for recovery of the lactate in both cases.

According to yet another aspect, the present technology can include a biorefinery system for optimizing a simultaneous or interlinked production of lactate or Small Chain Fatty Acids (SCFA's) and mannitol. The system can include a first reactor pool, a second reactor pool and a recirculation line. The first reactor pool can be configured for production of lactate or SCFA's. The second reactor pool can be configured for production of mannitol using rumen bacteria as biocatalysts in such a way that after recovery of the mannitol a residual process fluid is applied to the first reactor pool for further elevating both the lactate or SCFA's and the mannitol levels of a biorefining as a whole. The recirculation line can be configured for transferring at least a portion of the residual process fluid exiting the second reactor pool from a second end for introduction back into a first end of the second reactor pool opposite to that of the second end. The second reactor pool can be in direct communication with the first reactor pool.

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, methods and systems for carrying out the several purposes of the present technology. It is, therefore, that the claims be regarded as including such equivalent constructions insofar as they do not depart from the spirit and scope of the present technology.

It is therefore an object of the present technology to provide a new and novel method and apparatus for the utilization of zero fiber and other side streams that has all of the advantages of the known mannitol production systems and methods and none of the disadvantages.

It is another object of the present technology to provide a new and novel method and apparatus for the utilization of zero fiber and other side streams that may be easily and efficiently manufactured and marketed.

An even further object of the present technology is to provide a new and novel method and apparatus for the utilization of zero fiber and other side streams that has a low cost of manufacture with regard to both materials and labor, and which accordingly is then susceptible of low prices of sale to the consuming public, thereby making such method and apparatus for the utilization of zero fiber and other side streams economically available to the buying public.

Still another object of the present technology is to provide a new method and apparatus for the utilization of zero fiber and other side streams that provides in the apparatuses and methods of the prior art some of the advantages thereof, while simultaneously overcoming some of the disadvantages normally associated therewith.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof. Such description makes reference to the annexed drawings wherein:

FIG. 1 is a flow chart of an embodiment of the method of utilizing intestinal bacteria for increasing productivity or production levels of desired biotechnical products constructed in accordance with the principles of the present technology.

FIG. 2 is a schematic picture and case example of the present technology based on the pilot experiments in Hiedanranta biorefinery plant in Tampere, Finland.

FIG. 3A is a photograph of the production of hydrogen as biohydrogen from the process broth. The bubbles of hydrogen can be collected with a funnel above the liquid surface of the fluid.

FIG. 3B is a graph showing a concentration of hydrogen analyzed from the funnel space of 20 m³, above the fermentation broth (see the graph). Hydrogen can be used as an energy gas or reducing agent in the industries. Also various traffic use is possible.

FIG. 4 is a graph showing the eight run with the Pilot equipment in Tampere in February 2020.

FIG. 5 is a graph showing laboratory experiments for the mannitol production from Hiedanranta, Tampere trials.

FIG. 6 are graphs showing results of experiments conducted in laboratory from Test 1 of Tests 1-3.

FIG. 6 are graphs showing results of experiments conducted in laboratory from Test 1 of Tests 1-3.

FIGS. 7A-7F are graphs showing results of experiments conducted in laboratory from Test 2 of Tests 1-3.

FIGS. 8A-8I are graphs showing results of experiments conducted in laboratory from Test 3 of Tests 1-3.

FIG. 9 are graphs showings the results of experiments conducted in Hiedanrante, Tampere, Finland from Test 7.

FIG. 10 are graphs showings the results of experiments conducted in Hiedanrante, Tampere, Finland from Test 8.

FIG. 11 is a flowchart of the biorefinery process of the present technology.

The same reference numerals refer to the same parts throughout the various figures.

### DETAILED DESCRIPTION OF THE EMBODIMENTS

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

Slaughter waste normally contains considerable amounts of so-called stomach manure. Its biotechnical treatment rapidly decreases the waste problem. With the help of the method and device according to this invention stomach manure is first used as a biocatalyst in a process that produces mannitol. After that the lactic acid that at the same time farms into the slaughter waste is converted with the help of microbes into propionic acid or other useful compounds.

When mannitol is produced with microbes, lactic add bacteria are normally the production organisms that are used. They are known safe production organisms, but it can be challenging to find a suitable strain for an efficient enough conversion of fructose to mannitol.

Using a method according to this invention, fructose or waste rich in fructose, a byproduct or a supplement is added into slaughter waste. This material contains for example at a bovine slaughterhouse a large amount of so-called stomach manure which, in practice, is the content of the rumen and other stomach content of the slaughtered animals. About 50 kg of this is formed per animal.

With the help of stomach manure microbes or other microbes added to them, also other different waste materials that are integrated into the process, can be utilized.

With the help of their common metabolism the microflora of the rumen that is contained in the stomach manure is capable of changing into mannitol the fructose that is in the waste or byproduct or added into it. At the same time the lactic acid bacteria form lactate which lowers the pH-value of the process liquid. With the help of certain lactic acid bacteria and other bacteria, such as Lactobacillus xylosus, Propionibacterium shermani and Propionibacterium acidipropionici lactate can be converted into propionate, i.e. propionic acid. The Clostridium propionicum bacteria form propionic acid, acetate, ammonia and carbon dioxide from L-alanine. The optimal pH for the formation of propionic acid is often 6,5. In addition to these products mentioned above, also several other organic and inorganic compounds are formed in the process. The ammonium salts formed in it can be precipitated apart from the process liquid and collected for use as a fertilizer together with the residue of the process liquid, or it can be separated from it. Thus with a method according to this invention fertilizers may be gained from stomach manure or other waste.

The mixed microbe population or mixed culture of the rumen can also convert glucose found in waste material or byproduct or other raw material, into mannitol. This requires that glucose is first enzymatically converted into fructose. in this case the same glucose-isomerase enzymes that are used in the production of fructose syrup (HFCS, high fructose corn syrup, GFS, glucose-fructose syrup, high-fructose maize-syrup, glucose/fructose) can be used.

In practice HFCS is produced from raw material that is rich in starch, mainly from corn. Also many other plants such as potato and cereal contain significant amounts of plant starch. The following enzymes participate in the reaction:

1. Alpha-amylase (forms short chain oligosaccharides from starch)

2. Glucoamylase (splits oligosaccharides into glucose)

3. Glucose-isomerase (converts about 42% of fructose and 50-52% of glucose and some other sugars into the mixture)

Using a method according to this invention fructose is produced from glucose if necessary with the help of industrial enzymes presented above or with the help of other industrial enzymes that release glucose. For example with the help of lactic acid bacteria mannitol can further be obtained from fructose. With the help of this process, the added value of many biomasses, side streams and waste materials can thus be increased. Glucose is a molecular structure that is commonly present in natural materials such as starch, cellulose and hemicellulose. Fructose is found e.g. in the wastes and side streams of sugar, fruit and berry industries. Naturally it is clear that in this case microbe cells or other cells, tissue, cell parts or similar may function as biocatalysts, in addition to or along with enzymes or as sections that replace them.

Because the potential harmfulness of extensive use of fructose has been raised in recent discussion related to health, nutrition and chronic illnesses, it is advantageous to biotechnically refine fructose further to another useful form. Contrary to for example glucose, fructose does not metabolize in all the cells of the body, only in the liver. If excessive amounts of fructose are used this involves e.g. obesity and abdominal obesity risks and also the risks of fatty liver and other liver diseases. The energy content of mannitol is fairly low compared to fructose and glucose and many other molecules but it has considerable value of use e.g. in lozenges, chewing gums, medicinal products, pastries etc. as an additive and sweetener that improves product quality, such as taste, freshness etc. Also in this respect it is justified to convert fructose, glucose and other sugars to mannitol.

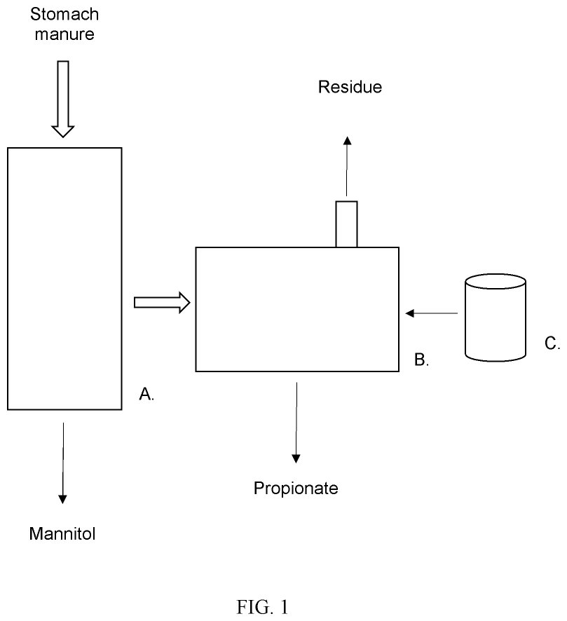

When sucrose (consists of glucose and fructose) was added into slaughter waste (stomach manure etc.), large amounts of mannitol formed into it. When the pH was not tried to be adjusted or kept at a certain pH level, contrary to what is normally advantageous in biotechnical processes, the yield was close to the theoretical maximum level being approximately 18 mg/ml. When the pH level was adjusted to be between 5 and 6,5, the corresponding production level was only 3 mg/ml. The high level of mannitol that occurred when the pH level was not adjusted during the biotechnical process was an unexpected reaction of the normal microflora of the rumen. A model for the implementation of the production process of mannitol is presented in FIG. 1.

If, as described above, the pH was not tried to be adjusted or stabilized, considerable amounts of lactate, i.e. lactic acid formed into the process liquid. An advantageous means of exploiting this is to convert it with the help of lactic acid bacteria into propionate which has a high energy content and which may be used as silage or feed for animals kept for fur or as food for other animals or even as an additive or preservative for human nutrition. It can also be used to prevent the deterioration of timber and structures and the icing of road surfaces.

An alternative way to process biomass based suspension or liquid is anaerobic acetone-butanol fermentation. The growth of clostridia can be accelerated by leading carbon dioxide into the gas solution or process liquid. Because clostridia can withstand up to 100% carbon dioxide content while growing a culture, this can be used to speed up the reaction. Clostridia can also be used to produce other organic acids such as butyric acid and valeric acid.

An installation to exploit the method according to this invention consists of A. a mannitol reactor, B. a production pool for propionic acid formation and C. a seed fermenter (FIG. 1).

A method of utilizing intestinal bacteria, the method comprising the steps of: preparing a process liquid by adding a sugar selected from the group consisting of monosaccharides, disaccharides, and their polymers into manure selected from the group consisting of stomach manure of a slaughtered animal, manure produced by an animal, and refinery sludge; and increasing productivity or production levels of desired biotechnical products in the process liquid.

In one or all aspects, the present technology can include a method of producing products from manure including the steps of adjusting a humidity percentage of manure in a reactor. Then preparing a process liquid by adding a sugar selected from the group consisting of monosaccharides, disaccharides, and their polymers to the manure after the humidity percentage of the manure has been adjusted, the manure being selected from the group consisting of stomach manure of a slaughtered animal, manure produced by an animal, and refinery sludge containing manure. The manure being not from a human. Converting at least the sugar added to the manure into mannitol using lactic acid bacteria in the manure, and then removing the mannitol from the process liquid. After which, adjusting a pH value of the process liquid by adding a base to the process liquid after removal of the mannitol for production of propionic acid, and then increasing productivity or production levels of desired biotechnical products in the process liquid.

In some or all embodiments of the present technology, the sugar can be sucrose, glucose, fructose, and a mixture of any of sucrose, glucose and fructose.

In some or all embodiments of the present technology, the sugar can be glucose and fructose that is produced enzymatically as a result of decomposition of starch, cellulose, hemicellulose or other macromolecule.

In some or all embodiments of the present technology, an amount of the fructose can be added by enzymatic means.

Some or all embodiments of the present technology can include the step of converting the sugar in the manure into mannitol using lactic acid bacteria in the manure.

Some or all embodiments of the present technology can include the step of lowering a pH value of the process liquid, prior to removing the mannitol, by changing the glucose into lactic acid by at least the lactic acid bacteria and microbes performing acetic acid fermentation, which simultaneously prevents decomposition or metabolization of the mannitol that has formed into the process liquid.

In some or all embodiments of the present technology, the pH value can start to be raised by adding a base when at least most of the fructose has converted into mannitol.

Some or all embodiments of the present technology can include the step of removing the mannitol by crystallization prior to raising the pH value.

Some or all embodiments of the present technology can include the step of heating the process liquid by leading a gas emission containing one of CO and CO₂ into the process liquid, wherein the gas emission is formed from a combustion reaction.

Some or all embodiments of the present technology can include the step of converting the lactic acid formed by the acetic acid fermentation to propionate.

In some or all embodiments of the present technology, the propionate can be propionic acid, and wherein Clostridium bacteria is used in converting the propionic acid into an organic acid or an alcohol.

Some or all embodiments of the present technology can include the step of adding at least one bacterial strain into the manure for assisting in changing a part of the lactic acid into the propionate, wherein the part of the lactic acid is collected for burning or for chemical utilization as a preservative in secondary products.

In some or all embodiments of the present technology, the sugar can be added to the manure after adjusting a humidity percentage of the manure in a reactor.

Some or all embodiments of the present technology can include the steps leading a residue of the process liquid into a container after removing the mannitol; and adjusting the pH value and temperature of the process liquid in the container for the production of propionic acid.

Some or all embodiments of the present technology can include the step of adjusting microbial levels of the process liquid using microbial inoculums that is inoculated from an inoculation fermentor, wherein the inoculation fermentor has a dosing pump.

Some or all embodiments of the present technology can include the step of precipitating ammonium salts formed from the manure in the process liquid to fertilizers.

Some aspects of the present technology can include an apparatus for utilizing intestinal bacteria. The apparatus can include a reactor having a configuration capable of containing a process liquid including manure and a sugar, and adjusting a humidity percentage of the manure in the reactor. A container can be included that is configured to receive a residue of the process liquid from the reactor, and adjusting a pH value and temperature of the residue suitable for production of propionic acid. An inoculation fermentor can be included with a dosing pump, wherein the inoculation fermentor can have a configuration capable of inoculating microbial inoculums therein, and wherein the dosing pump has a configuration capable of pumping the microbial inoculums to the reactor.

In biotechnical processes using biocatalysts, it is essential to implement the simultaneous planning strategies both for the growth and maintenance of the biocatalysts and for their reactions, as well as for the hardware design and adjustments of the bioreactors. The essential feature of this invention is to synchronize the biochemical and microbiological process with the design of the bioprocessing plant. These ideas have been tested in several pilot experiments, where both enzymes and microbial mixed cultures have been used in the pool-shaped reactors.

In the exemplary, the present technology can include the lowering pH (by the lactic acid bacterial population), preventing the degradation of mannitol produced partially by the same bacterial population. This could be seen in following laboratory experiments. There the mannitol is decreasing when the pH is rising. Therefore, further measures may be needed to ensure the simultaneous high levels of the two chemicals (lactate and mannitol). This is because the excessive lactate could lower the pH too much, leading to other restrictions of the mannitol process or possibly to alternate pathways for the sugars.

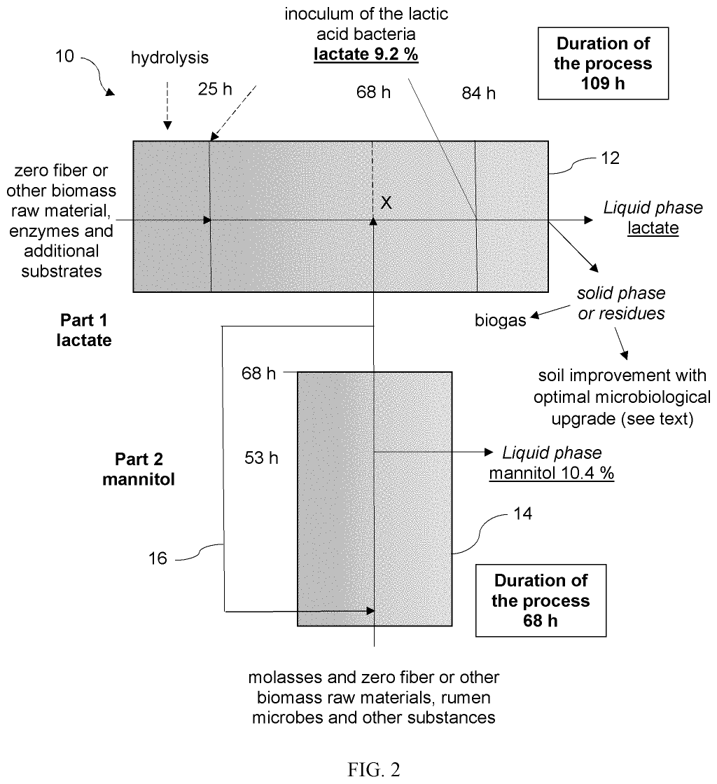

The pool construct allows the bioprocess fluid or broth or suspension to be moved forward while it is processed or adjusted. It is also easier to carry out the measurements of the process parameters alongside the progress within the reactors, or during the succession of the biochemical or microbiological reaction sequences. In the present invention, the pool shaped reactor system 10 is illustrated in FIG. 2 including a first reactor pool (pool 1) 12, a second reactor pool (pool 2) 14 and a recirculation line 16.

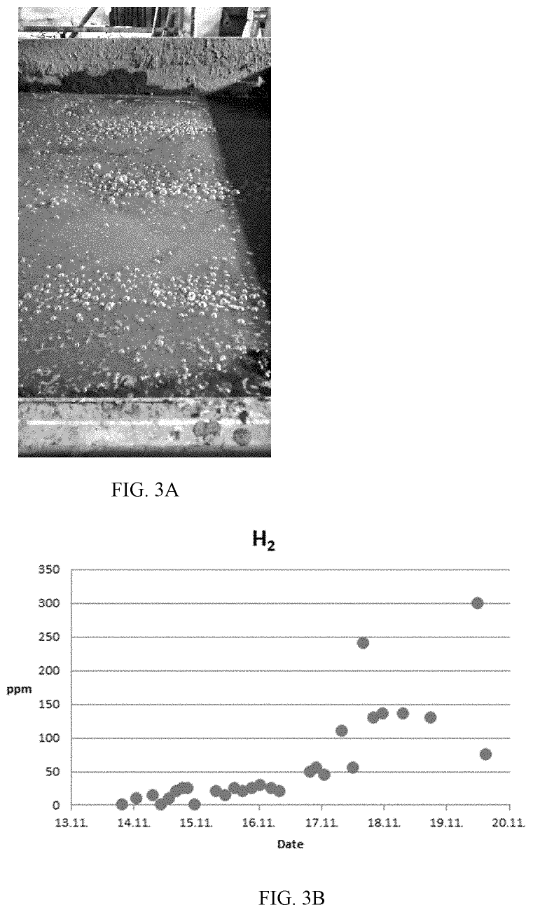

This flexibility of the process control is important, not only for the timely recovery of chemical products but also for the collection of gaseous substances. For example, the hydrogen gas can be formed during a specific phase of the process. If the oxygen content of the fermentation broth is low enough, this leads to the formation of butyrate and hydrogen (FIGS. 3A and 3B).

If the organic acids, such as lactate and propionate, are produced microbiologically from the slaughterhouse waste or from the potato industry waste, some members of the normal flora or the additional industrial strains of Clostridium pasteurianum could facilitate the formation of valeric acid (Den Boer et al. 2016; Schwede et al. 2017). The formation takes place as a consequence of the condensation reaction between lactate and propionate. Besides, the Clostridium pasteurianum strains or its closest relatives are strictly anaerobic bacteria which also produce hydrogen gas (H₂), and bind atmospheric nitrogen (N₂) in an autonomous fashion (Hakalehto 2016b). In fact, it has been proven out that the lactic acid bacteria can boost the onset of clostridial growth by their CO₂ production (Hakalehto and Hänninen 2012; Hakalehto 2015a). This could make it possible to combine the production of H₂ (for hytane gas mixture, for example) with the conversion of organic wastes into useful chemicals, such as organic acids (lactate, propionate, butyrate, acetate, valerate etc.) or 2,3-butanediol, butanol or ethanol, as well as with the production of sugar alcohols, such as mannitol, xylitol or sorbitol. This could lead to a biorefinery process, which could in the same or parallel units facilitate the production of

1. energy gases,

2. valuable chemicals, and

3. organic fertilizers or soil improvement agents,

in the process unit with one or several industrial strains which could function together with the natural microflora derived from the side stream in question. The above-mentioned microbiological method to upgrade the residual fraction by autonomous Nitrogen-fixing bacteria could remarkably improve the economics of the zero fiber processing, or alternatively that of any other biomass processing multi-strain or CBP-type of bioprocess. This could produce huge savings in:

A. investment costs, as the production unit volumes go down,

B. energy efficiency, as the power source is within the process,

C. adjustments and control, which can be handled on the ecosystem level at best,

D. removing at least a part of the gate fees in the treatment of the residual fraction and by bringing an important economical value for it

in the said manufacturing unit. The corresponding and required technologies could make it possible to learn to adjust the process for the numerous goods (gases, chemical commodities, fertilizers) according to the economic conditions and the demand in the market. Consequently, it is possible to build up a multipurpose biorefinery unit (MBPU) with low investment costs. It can obtain energy gases (hydrogen, methane, hytane) or electricity from the process itself. Such MBPU process, however, may also need clever partitioning of the process or unit operations.

Different organic materials and side streams can be produced in the MBPU. Besides the residual fractions of the forest, potato or slaughterhouse industries, also different agricultural or forestry wastes, as well as side streams of the sugar or brewing or fruit processing industries could be considered as potential raw materials. Since most of these raw materials consist of organic polymers, their hydrolysis is required. This could be carried out by acid or base, or by hot steam or water, or by some other physicochemical methods, as well as by enzymatic hydrolysis. In the latter kind of process, temperature changes could be utilized for improving the yield from the hydrolysis. The raw materials for the unit could include many other biomass sources besides the zero fiber, such as agricultural wastes, fruit waste, food industry wastes, sugar industry waste etc.

In addition to the two SCFA's (Small Chain Fatty Acids), lactate and propionate, it is possible to produce a third one, namely butyric acid (butyrate). This has been formed in the process utilizing the zero fiber and paunch as raw materials. It has been proven in our earlier studies that the CO2 emitted by lactic acid bacteria provokes and speeds up the growth of butyric acid clostridia (Hakalehto and Hänninen 2012, Hakalehto 2015a).

Typically for the production of the SCFA's their formation is peaking in the anoxic conditions. If the pH is around 6.5, the main product of the mixed fermentation is often propionate, at the pH of 5.5 it is butyrate, and at the pH of 4.5 acetate. Lactate is converted into other SCFA's (Hakalehto 2015b). The production of propionate, for example, can also get performed by a food-grade micro-organism Propionibacterium acidipropionici, which is accepted for food production by EFSA (European Food Safety Association).

In order to carry out the CBP type of reaction, one has to support it or at least is obliged to suppose that the conditions for the enzymatic hydrolysis will remain allowable during the accompanying microbial process. In turn, the continuous hydrolysis keeps the conditions ideal for microbial metabolism as it limits such regulatory functions as feedback inhibition for itself. Therefore, it is beneficial for the outcome, productivity and yield of the process to ensure the incessant enzymatic function in the production broth, as well as the boosting up of the desired microbiological reaction in a mixed metabolism situation. In fact, the CBP process is often easier to be converted into a continuous process. However, if the products are mixed or variable ones, the processing plan may include several reactor, tanks or pools for various partial processes or phases.

In practice, the challenges of the CBP often relate to the diffusion reactions, which means in practice that gradients or different zones are easily formed into the process. This is more likely in the big units. On the other hand, these gradients could also be advantageous for the process outcome, productivity and yield, provided that the gradients can be controlled well enough. Therefore, with the intention of

A. arranging suitable conditions throughout the reactor broth for both enzymatic hydrolysis and the microbial process, and

B. controlling the gradients related to various reactions

the equipment and method according to the present invention offers means to exercise such activities when pursuing the multi-strain or CBP-type of reaction in a biorefinery or equivalent. In a big production unit a pool-type of reactor if often advisable for the improved options of control and sequential process mode. According to the present invention, it is possible to monitor and measure such parameters as temperature, pH, turbidity, concentrations of various gases, conductivity, pO₂, pCO₂, impedance, viscosity, glucose or fructose content or any other parameter. These measurements can be taken from the process broth moving on by the rotors, propellers, liquid blows, screws, paddlewheels or equivalent. The measurement can be taken from any point of the process, and the result can be used for the adjustments or for planning of the additions. It is also possible to move the process fluid from one point to another by pumping systems.

We have carried out the processing of slaughterhouse wastes (paunch and other fractions) together with molasses (US Patent Application (US20160251684A1) (Hakalehto 2016c)). In these cases the fructose of the molasses is converted into mannitol. When the molasses are added to the residual “zero fiber” fraction of the pulp and paper industries, this leads to the formation of organic acids, particularly lactic acid (Beckinghausen et al. 2019). Moreover, if paunch and molasses are added to this side stream, this also leads to the accumulation of mannitol in the favorable conditions in the multi-strain process.

In an advantageous mode of processing various wastes into mannitol and lactate, or into other organic acids, a mixed microbial culture of rumen bacteria can be used as the biocatalyst. This approach can be performed according to the procedure of the US Patent Application (US20160251684A1) (Hakalehto 2016c) These processes can be carried out simultaneously, namely the lactate and mannitol production, in the one and same reactor system. However, according to the present invention, the optimal process mode is a partially separated system of two pools (FIG. 2) and first reactor pool 12 and a second reactor pool 14.

One important aspect is the difference in the composition of the LAB microflora. The flora in the lactate production phase (out of the hydrolyzed cellulose) have the optimal temperature of 28-32° C., whereas the mannitol production is carried out by strains selected at 35-40° C. The former process takes about 90-100 hours to reach maximal production rate, and the latter one about 50-70 hours for the same level.

Referring to FIG. 3A, the photograph of the production of hydrogen as biohydrogen from the process broth can be seen as bubbles. The bubbles of hydrogen can be collected with a funnel or any other containment system above the liquid surface of the fluid.

The graph in FIG. 3B shows a concentration of hydrogen analyzed from the funnel space of 20 m³, above the fermentation broth. Hydrogen can be used as an energy gas or reducing agent in the industries. Also various traffic use is possible.

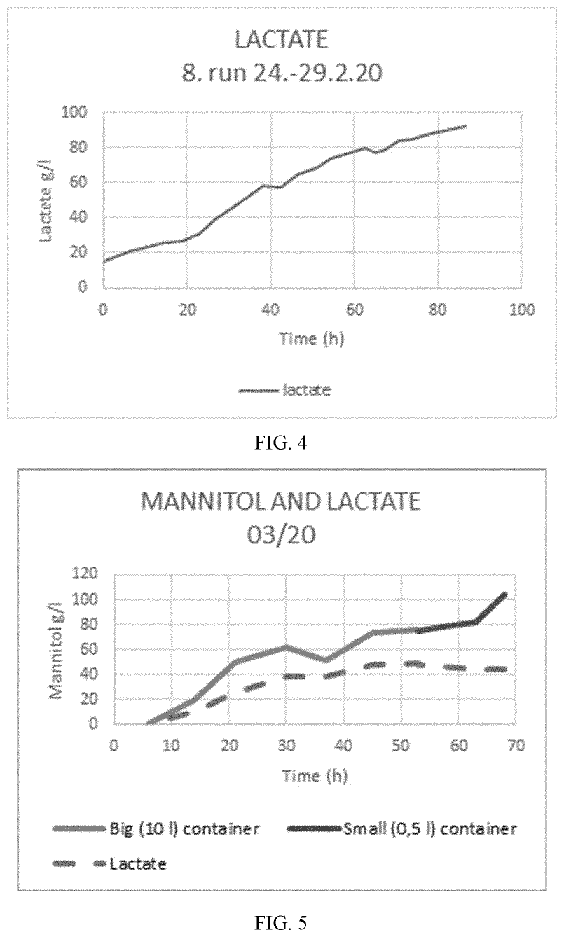

However, in the large-scale treatment of e.g. cellulosic waste combined with molasses, it turned out that the lactate process (FIG. 4) is at least 20 hours more time-consuming in reaching the metabolic completion than the mannitol process (FIG. 5). In this context the term “metabolic completion” refers to the maximal yields of the mixed fermentation. The two processes support each other:

1. The accumulation of lactate supports the mannitol process, as the lowering pH protects and preserves the product mannitol.

2. The mannitol process outcome is beneficial for the last stages of lactate production, as the residual fraction after the recovery of the product mannitol is combined with the final stages of the lactate production for boosting the production rate and product yield. Then it is possible to elevate the temperature from 28-32° C. to 35-40° C.

It can be seen from the graph in FIG. 4 that the lactate production curve is in a continuous upward direction. Consequently, the optimal processing time is at least 90 hours, which is over 20% more time that is required for the mannitol production maximum to be reached. Therefore, the advantageous arrangement of the two fermentations, namely the lactate and mannitol processes, would be starting them in separate pools as illustrated separately. Later on the remaining fraction after the removal of mannitol could be integrated into the lactate production. Parts of that fraction could also be returned back to the beginning of the mannitol process. The data in FIG. 4 was obtained from an eight run with the Pilot equipment in Tampere, Finland on February 2020.

FIG. 5 is a graph showing laboratory experiments for the mannitol production from Hiedanranta, Tampere trials. The elevated production levels were achieved by boosting the process using low-cost fructose syrup. Thus, the process broth could be added to the lactate process after the removal of mannitol, to boost that second process.

It is also noteworthy, that in lowered oxygen content, more butyric acid and hydrogen can be formed.

This synergism of two separated reaction is optimal and effective only when the processes are synchronized with the main processes starting in separate reactors or tanks or pools but to be combined in a delicate way as illustrated here (FIG. 2). Consequently, we developed and tested the method, by the teachings of the present invention by which the lactate fermentation and mannitol production are started in different reactors or pools, and then the broths of the two reactors are combined as instructed here. At this point, the mannitol is often preferably removed from the corresponding process fluid. It can also be added within the entire process fluid to the pool number 1, but then the total volume will increase very large. These reactions can be of the CBP-type with the enzymes still active in the broth.

After the mannitol production has reached its maximum, and the product recovered, for example by a separate reactor for crystallization, or by a series of reactors, the remaining active biological fluid can be added to the lactate production unit and into the lactate fermentation broth. There it can boost the lactate production. During the mannitol production, the initial lactic acid bacteria (LAB) originating from the rumen contribute to the preservation of mannitol by keeping the pH low (Hakalehto 2016c). The division and initiation of the two processes in two reactors increase the production of both of the processes, as they can be adjusted and optimized separately for the beginning. However, it is advantageous to combine the residual fraction of the mannitol process into the ongoing lactate production, which brings also other synergistic benefits that can be achieved by this combination. Moreover, lactate is one of the main natural product of the rumen LAB, which, besides the stabilization of mannitol, also can be collected as a by-product from that process (pool number 2).

### EXAMPLE 1

In the industrial piloting of lactate production from the zero fiber, 600 liters of the cellulolytic material was treated with 1000 g of Viscamyl Flow and 750 g of Optidex enzymes. The hydrolysis phase prior to the microbial process lasted for 25 hours. For the hydrolysis 300 liters of water was added, 50% of which was obtained from the residual fraction of the previous runs. For the microbial inoculum, 51 kg of rumen biomass and 7 kg of sour milk were added 20 hours after the onset of the fermentation phase. Also 175 kg of molasses were added, together with the microbes and 65 liters of NaOH (40%) and 17 kg of CaCO₃ for the pH adjustment during the process, as well as 21.5 kg of meat bone meal. The volume of the process water was increased by 127 liters during the process run. The pH was kept between 5.1-6.5 by the addition of NaOH, and the temperature was 30° C., which favored the lactic acid bacteria derived from the lake. The steadily increasing lactate production is presented in FIG. 4. The results of this experiment indicated steady growth of lactate concentration during the experiment, reaching 9.2% in the end.

### EXAMPLE 2

In the simultaneous production of mannitol and lactate in the laboratory, the focus was in the optimization of the former substance, since the optimization of the lactate as a product was carried out as described in the Example 1. The mannitol production was boosted for the last quarter of the process run by adding some fructose syrup to the broth. The temperature for the hydrolysis was 40° C., and it was 37° C. for the mannitol fermentation. Ten liter buckets were used as containers or reaction vessels.

The hydrolysis phase took 12 hours, and the enzymes “Viscamyl Flow” (2 g) and “Optidex” (1.4 g) were added to the suspension of 1.5 liters of zero fiber (or some corresponding cellulolytic substrate) with 0.5 liters of water. For the following microbial inoculation, 3.5 liters of rumen contents or paunch were added to the container together with 2.1 kg molasses, 500 g of meat bone meal (mbm) and 100 g of liver. The pH adjustment during the process was carried out with 70 ml NaOH (40%) and 300 g CaCO₃, Up to 5 liters of water was added during the process. Regardless of the extensive dilution, the mannitol concentration reached 10.4% and lactate concentration elevated close to 5% without optimization. The hydrolysis can continue as the CBP reaction during the microbiological process.

In both Examples, the metabolites were monitored using NMR (Nucleic Magnetic Resonance) method (Laatikainen et al. 2016). These results indicated that after the completion of the mannitol process, the broth still contained glucose and mesophilic lactic acid bacteria. Their addition to the ongoing lactate fermentation in another reactor or pool could add the final yield particularly at the elevated temperature (30->37° C.).

In the mannitol process no more than 5% of the lactic acid was produced, whereas the production level in pool 1 (12) was 9.2%. After the removal of mannitol, the residual fraction could induce higher lactate yields at 37° C. when added to pool 1 (12) from pool 2 (14). This could be deducted also from the relatively high level of glucose present in the broth according to the NMR (about 0.5%) in the end of the process. This indicates the potential of the microbial culture to elevate the lactate production during the remaining phase.

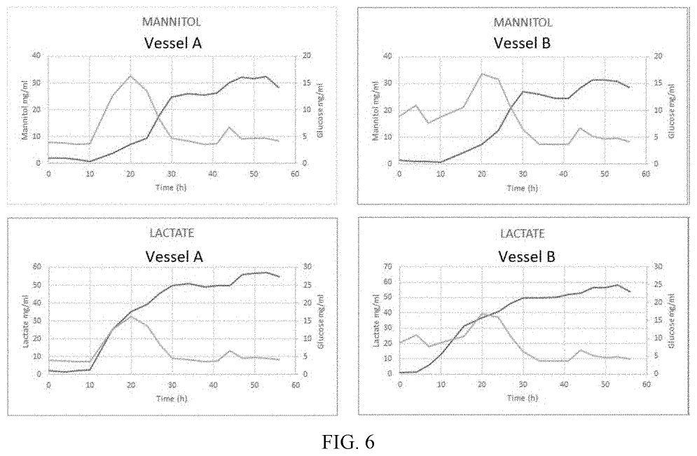

In support of the above description and the present technology, FIGS. 6-8I shows the results of laboratory experiments (Tests 1-3) for the production of mannitol in Vessel A (pool 2) and lactate in Vessel B (pool 1).

In Test 1 (FIG. 6), it can be appreciated from the top graphs show the production of mannitol (mg/ml) represented by the blue line and glucose (mg/ml) represented by the grey line over time (hours “h”) in both reactor pools (Vessels A and B). Further regarding Test 1, it can be appreciated from the bottom graphs the production of lactate (mg/ml) represented by the blue line and glucose (mg/ml) represented by the grey line over time (hours “h”) in both reactor pools (Vessels A and B).

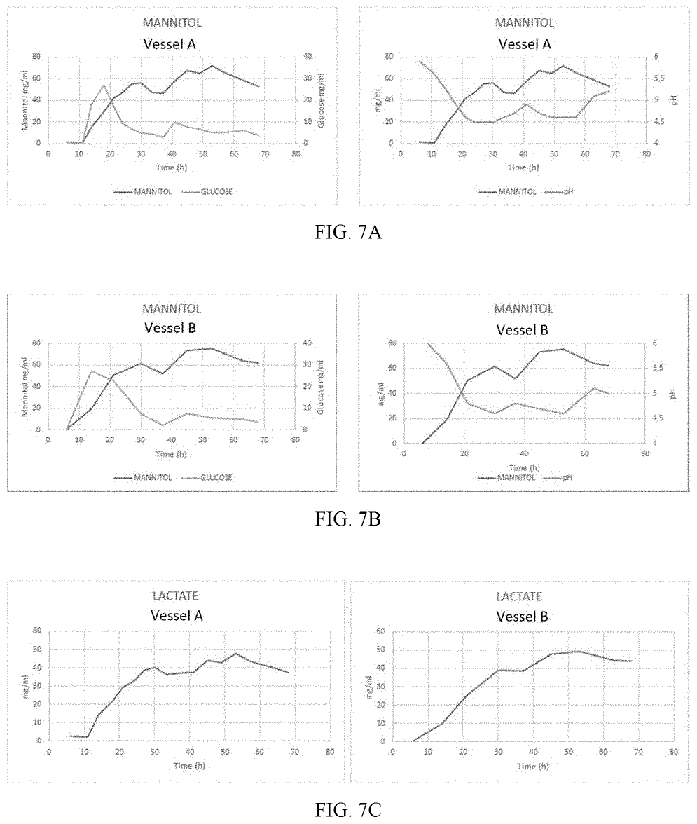

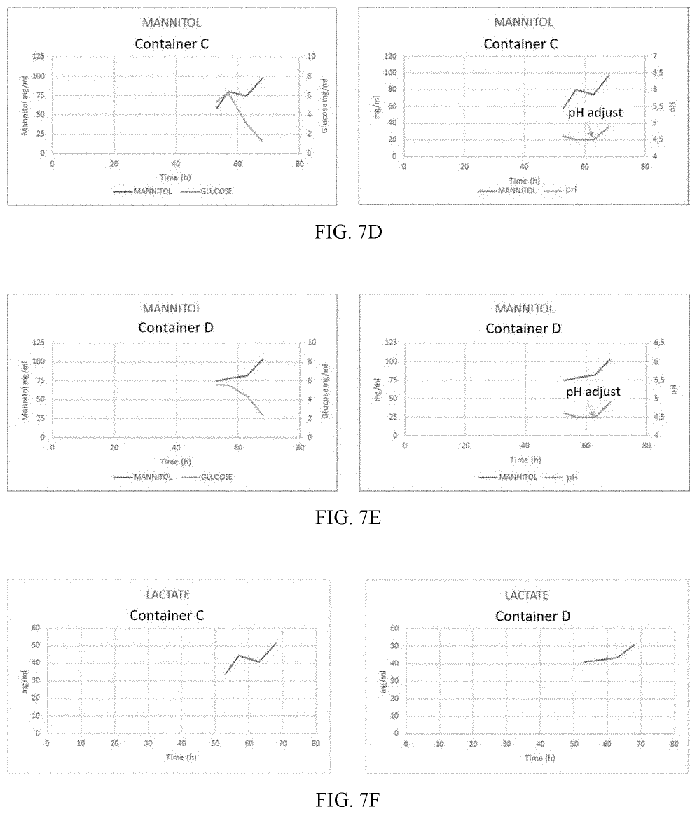

In Test 2 (FIGS. 7A and 7B), it can be appreciated from the graphs show the production of mannitol (mg/ml) represented by the blue line and glucose (mg/ml) represented by the grey line over time (hours “h”) in both reactor pools (Vessels A and B). The second set of graphs shows the production of mannitol (mg/ml) represented by the blue line with corresponding pH represented by the orange ling over time (hours “h”) in both reactor pools (Vessels A and B).

Further regarding Test 2 (FIG. 7C), it can be appreciated from the graphs show the production of lactate (mg/ml) represented by the blue line over time (hours “h”) in both reactor pools (Vessels A and B).

Further regarding Test 2 (FIGS. 7D-7E), it can be appreciated from the graphs the production of mannitol (mg/ml) represented by the blue line over time (hours “h”) in a container C. The second set of graphs shows the production of mannitol (mg/ml) represented by the blue line with a corresponding pH adjustment represented by the orange ling at predetermined time (hour “h”) in container C.

The graph in FIG. 7F, shows the production of lactate (mg/ml) represented by the blue line over time (hours “h”) corresponding to the time in FIGS. 7D-7E in container C.

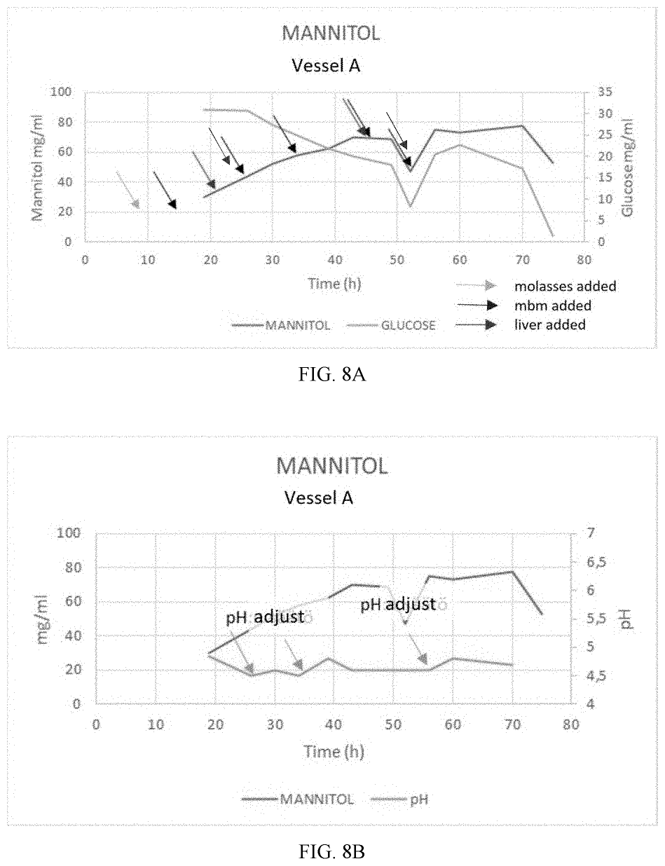

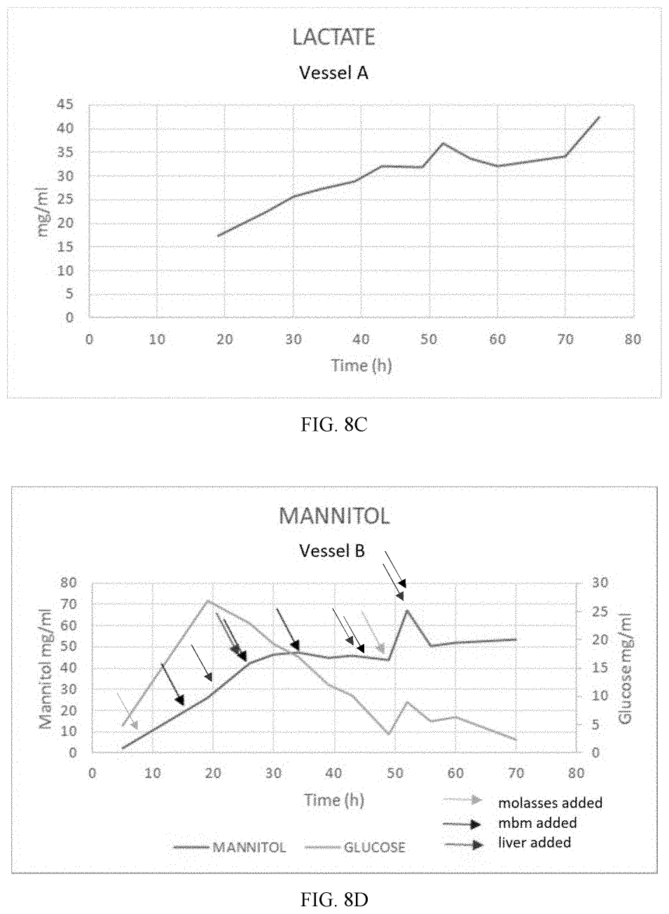

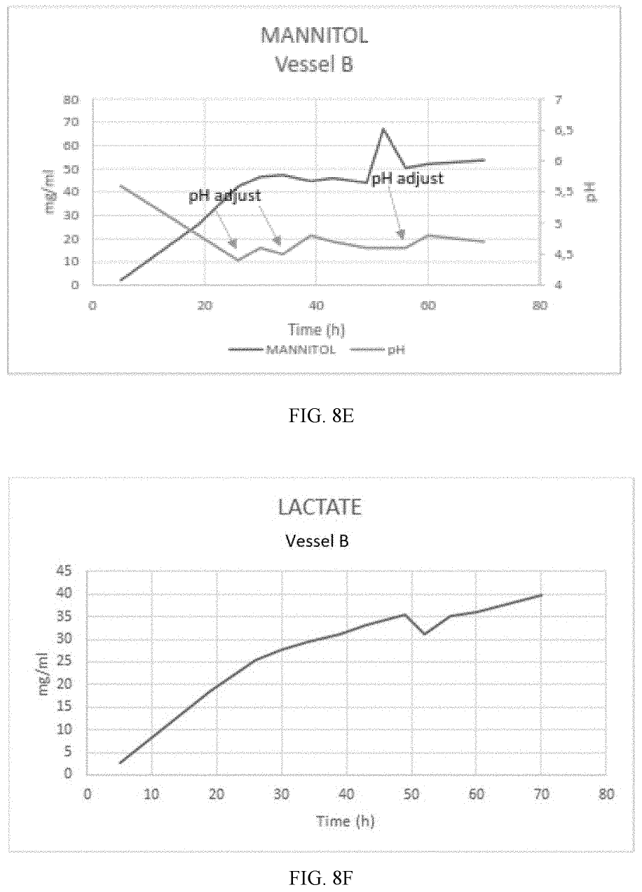

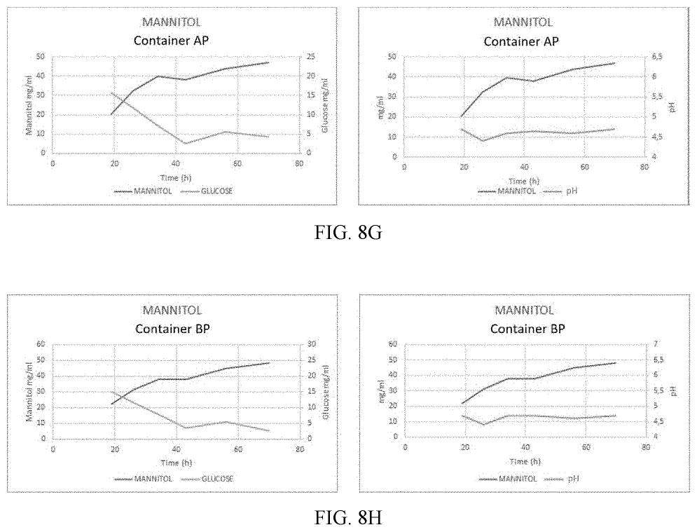

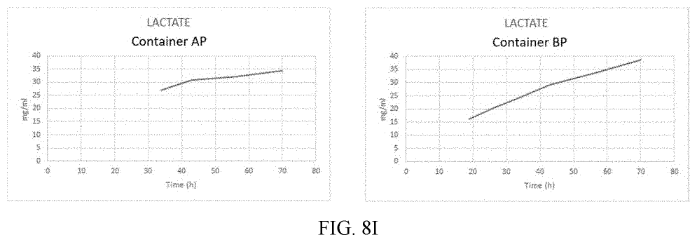

In Test 3 (FIG. 8A), it can be appreciated from the graph the production of mannitol (mg/ml) represented by the blue line and glucose (mg/ml) represented by the grey line over time (hours “h”) in Vessel A (pool 2). Further in Test 3, molasses, meat bone meal (mbm) and liver were added at predetermined times throughout the process. FIG. 8B shows the production of mannitol (mg/ml) represented by the blue line with a corresponding pH adjustment represented by the orange ling at predetermined time (hour “h”) in container C.

Further regarding Test 3 (FIG. 8C), it can be appreciated from the graph a showing of the production of lactate (mg/ml) represented by the blue line over time (hours “h”) in Vessel A (pool 2). While FIG. 8D is a graph showing the production of mannitol (mg/ml) represented by the blue line and glucose (mg/ml) represented by the grey line over time (hours “h”) in Vessel B (pool 1) with the addition of molasses, meat bone meal (mbm) and liver were added at predetermined times throughout the process.

FIG. 8E shows the production of mannitol (mg/ml) represented by the blue line with a corresponding pH adjustment represented by the orange ling at predetermined time (hour “h”) in Vessel B (pool 1). While FIG. 8F is a graph showing the production of lactate (mg/ml) represented by the blue line over time (hours “h”) in Vessel B (pool 1).

Still further regarding Test 3, FIG. 8G includes a graph showing the production of mannitol (mg/ml) represented by the blue line and glucose (mg/ml) represented by the grey line over time (hours “h”) in a container AP. The second graph shows the production of mannitol (mg/ml) represented by the blue line with a corresponding pH represented by the orange ling over time (hours “h”) in container AP.

FIG. 8H includes a graph showing the production of mannitol (mg/ml) represented by the blue line and glucose (mg/ml) represented by the grey line over time (hours “h”) in a container BP. The second graph shows the production of mannitol (mg/ml) represented by the blue line with a corresponding pH represented by the orange ling over time (hours “h”) in container BP.

With FIG. 8I, the graph shows the production of lactate (mg/ml) represented by the blue line over time (hours “h”) in the container AP and BP.

Further, FIG. 8I illustrates the lactate levels, which were rising but not too much, thus protecting the mannitol formed. This reactor broth could be combined with the broth in the principal lactate pool reactor to maximize the lactate yield. This would boost the lactate production there while the mannitol is already in the downstream processing phase.

The different process times in the graphs can be based on a discrepancy in temperatures between the two reactor pools. The temperatures of the two reactor pools were different, 28° C. in the “lactate pool” and 33-35° C. in the “mannitol pool”. Consequently, the lactic acid bacteria are of distinct origins mainly, from the lake bottom cellulosic sediments in the “lactate pool” and rumen contents in the “mannitol pool”. This temperature difference may have resulted in the different process times.

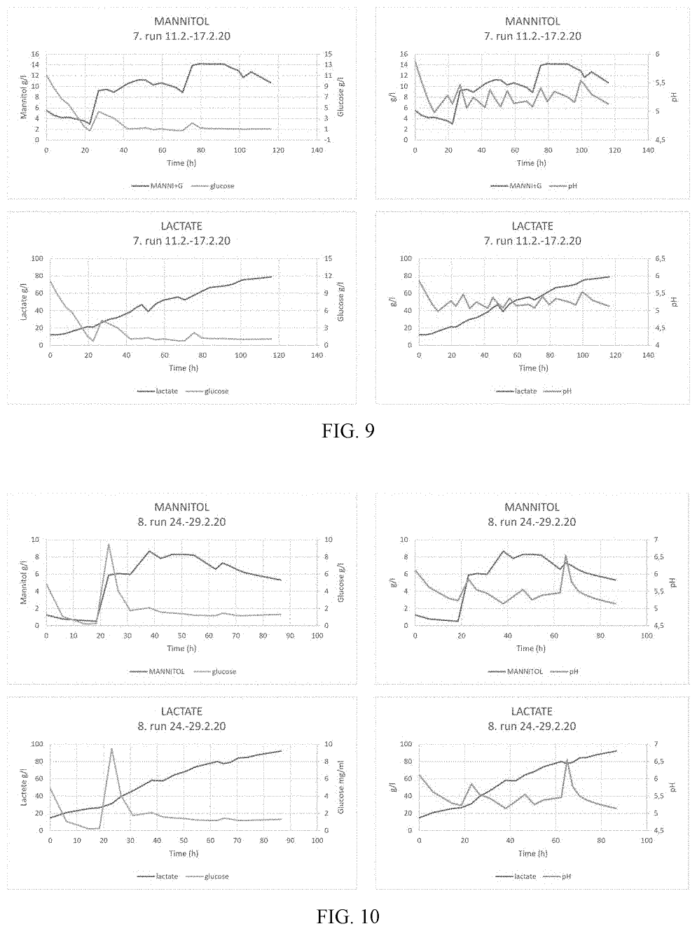

In support of the above description and the present technology, FIGS. 9 and 10 shows the results of experiments conducted in Hiedanranta, Tampere (February, 2020) with regard to Tests 7 and 8. Test 7 was executed by using zero fiber that was lifted from the lake bottom in January. 50% of test fiber was stored in a southern pile and 50% in a northern pile. Temperature of hydrolysis was 38° C. in the beginning and later 35° C.

After the hydrolysis temperature was lowered to 30° C. Meat and bone meal and chalk were added to the mass and its pH was risen to 5.9. 140 liters of end product of previous run and 5 liters of sour milk were added too. Gaseous Nitrogen was directed to the mass in short cycles to lower the oxygen content. Rumen microbes and molasses were added after 24, 36 and 72 hours form the beginning of the test. Meat bone meal, chalk and molasses were added during the test. pH of the mass was adjusted to 5.0-6.0 by adding NaOH.

In Test 7 (FIG. 9), it can be appreciated from the top graphs show the production of mannitol (mg/ml) represented by the blue line and glucose (mg/ml) represented by the grey line over time (hours “h”) and the production of mannitol (mg/ml) represented by the blue line and its corresponding pH represented by the orange line over time (hours “h”).

While the bottom graphs show the production of lactate (mg/ml) represented by the blue line and glucose (mg/ml) represented by the grey line over time (hours “h”) and the lactate of mannitol (mg/ml) represented by the blue line and its corresponding pH represented by the orange line over time (hours “h”).

In Test 8 (FIG. 10), it can be appreciated from the top graphs show the production of mannitol (mg/ml) represented by the blue line and glucose (mg/ml) represented by the grey line over time (hours “h”) and the production of mannitol (mg/ml) represented by the blue line and its corresponding pH represented by the orange line over time (hours “h”).

While the bottom graphs show the production of lactate (mg/ml) represented by the blue line and glucose (mg/ml) represented by the grey line over time (hours “h”) and the lactate of mannitol (mg/ml) represented by the blue line and its corresponding pH represented by the orange line over time (hours “h”).

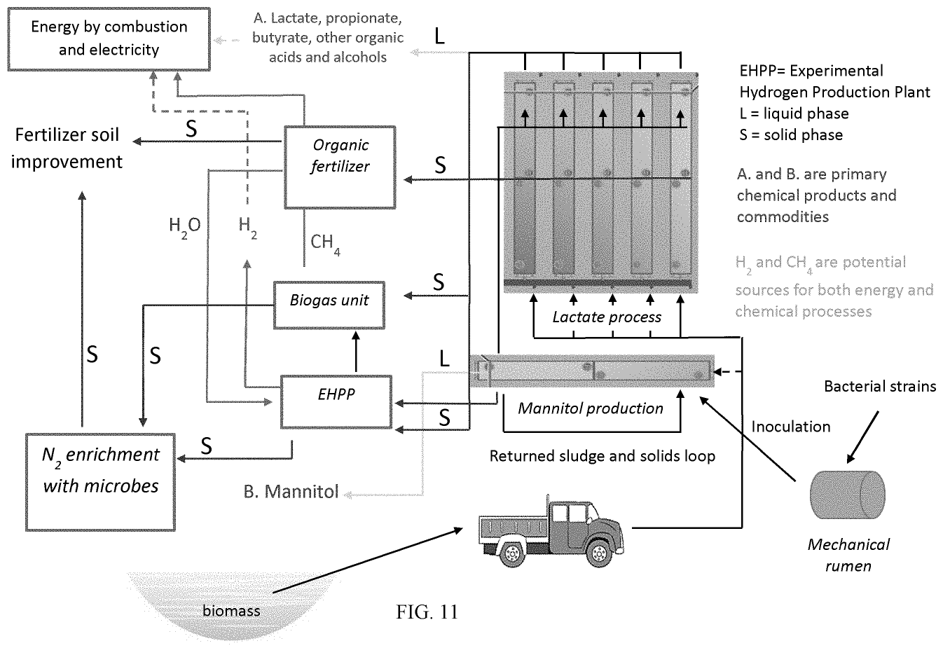

The present technology system, method and process can be shown as a flowchart, as best illustrated in FIG. 11. The biomass can be transported or provided to the lactate process of the first reactor pool. In the exemplary, the first reactor pool can include a manifold or distribution system that receives the biomass and provides it to multiple processing elements. A chemical product or commodity in liquid phase such as, but not limited to, lactate, propionate, butyrate, other organic acids and alcohols can be recovered from each of the processing elements of the first reactor pool.

Solid phase waste or materials from the first reactor pool can be collected/recovered and utilized as organic fertilizer or in the production of organic fertilizer. The solid phase waste or materials can be collected, recovered or extracted from the liquid phase lactate produced from the first reactor pool, and/or from each of the processing elements. Further, the solid phase waste or materials can be provided to a biogas unit for production of Nitrogen (N₂) enrichment with microbes.

The second reactor pool can receive inoculation, molasses, zero fiber or other biomass raw materials, rumen microbes and other substances for the bioproduction of mannitol. A portion of the mannitol can be provided to the first reactor pool at a specific location and/or process time. A chemical product or commodity in liquid phase such as mannitol can be collected, recovered or extracted from the second reactor pool. Additionally, the second reactor pool can include a returned sludge and solids loop that feeds material back to a beginning of the second reactor pool. Further, some of the biomass that is provided to the lactate process can be provided to the mannitol production of the second reactor pool.

An experimental hydrogen production plant (EHPP) can be utilized and configured to receive solid phase waste or materials from the first and second reactor pools, and to receive water (H₂O) from the production of the organic fertilizer. The EHPP can be configured to produce Hydrogen (H₂) and/or Methane (CH₄) that can then be utilized for combustion, energy production and other chemical processes.

The N₂ enrichment process or system can further be configured to receive solid phase waste or materials from the EHPP utilizable in the enrichment of N₂ that can then be used to improve or upgrade the organic fertilizer.

It can be appreciated that the present technology can be modular in nature, with specific processes and/or components added or removed in any arrangement to provide additional biproducts or efficiencies. For example, but not limited thereto, it can be evident that considerable amounts of organic substances with high energy contents, such as organic acids or alcohols, as well as energy-rich gases, such as Hydrogen and/or Methane, are emitted from the first reactor pool and/or the second reactor pool and/or from any byproduct resulting from the present technology.

Some of these high energy contents and energy-rich gases may evaporate in to the atmosphere. Consequently, it comes to mind that these fractions could collected and then utilized for combustion together with the residual liquid, suspension, or solids. Such an approach could add the liberation of energies in the incineration of the biomass. The incineration of the biomass can be implemented as a last approach of remove waste from the present technology process. A combustion unit can be utilized and configured to receive high energy contents and energy-rich gases from the waste in part as potential fuel in an incarnation combustion process. It can further be appreciated that the combustion unit may be utilized as part of an electrical, hot water or steam generation system, thereby utilizing bioproduct gases from the waste as fuel. Even further, the combustion unit may be utilized to preheat the biomass prior to entering the first reactor pool.

In another embodiment of the present technology, microbiological upgrading of the organic fertilizers obtainable from the residues of the MPBU can be accomplished utilizing a bacterial strain such as, but not limited to, one of the Clostridium bacterial strains. The use of the bacterial strains can upgrade the fertilizer byproduct to a higher nitrogen content, thereby increasing the fertilizer's effectiveness and marketability.

While embodiments of the method and apparatus for the utilization of zero fiber and other side streams have been described in detail, it should be apparent that modifications and variations thereto are possible, all of which fall within the true spirit and scope of the present technology. With respect to the above description then, it is to be realized that the optimum dimensional relationships for the parts of the present technology, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present technology. For example, any suitable sturdy material may be used instead of the above-described. And although synchronizing the biochemical and microbiological process utilizing a two reactor pool bioprocessing plant for the creating of mannitol and lactate have been described, it should be appreciated that the method and apparatus for the utilization of zero fiber and other side streams herein described is also suitable for the production of other Small Chain Fatty Acids (SCFA's) and mannitol.

Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

What is claimed as being new and desired to be protected by Letters Patent of the United States is as follows:

1. A method for optimizing a simultaneous or interlinked bioprocess for production of lactate or Small Chain Fatty Acids (SCFA's) and mannitol, said method comprising the steps of utilizing a first reactor pool for the production of lactate or SCFA's and a second reactor pool for the production of mannitol using rumen bacteria as biocatalysts in such a way that after recovery of the mannitol a residual process fluid is applied to the first reactor pool for further elevating levels of both the lactate or the SCFA's and the mannitol of a biorefining as a whole.

2. The method according to the claim 1, wherein inoculation of the first and second reactor pools is carried out simultaneously.

3. The method according to claim 2, wherein zero fiber or a cellulosic material in the first reactor pool is used as a main source of glucose in the first reactor pool.

4. The method according to claim 3, wherein cellulolytic enzymes are used for hydrolysis of the zero fiber or the cellulosic material, at least partially in a Consolidated Bioprocessing (CBP) mode, simultaneously with microbial processes.

5. The method according to claim 4, wherein fructose containing side streams are used as a raw material source for the production of the mannitol.

6. The method according to the claim 5, wherein the mannitol is recovered either from the second reactor pool into which the fructose and the rumen bacteria had been added, or from the first reactor pool if residues of the second reactor pool are transferred to the first reactor pool without prior recovery of the mannitol.

7. The method according to claim 6, wherein the cellulosic material being the zero fiber, is used in both of the first reactor pool and the second reactor pool amongst other raw materials.

8. The method according to claim 7, wherein purification of the lactate or the SCFA's is carried out of the residues of both of the first reactor pool and the second reactor pool either separately or as combined to each other.

9. The method according to claim 8, wherein hydrogen in a bubble flow is collected by suction for further use as an energy gas or a reducing agent.

10. The method according to claim 9, wherein a final fraction with solid particles or suspension is collected for soil improvement or organic fertilization purposes.

11. The method according to the claim 10, wherein the final fraction of the biorefinery is upgraded as soil improvement by using bacteria of the species Clostridium pasteurianum or other autonomously nitrogen-fixing species for increasing a soil nitrogen content available for plant growth.

12. The method according to claim 11, wherein the final fraction is used for replacing or increasing a soil humic fraction.

13. An apparatus for using the method as described in claim 12, wherein the first reactor pool and the second reactor pool are arranged into such a position with respect to each other that residues of the second reactor pool are added into the first reactor pool in a process phase that corresponds to a time point in the first reactor pool and process.

14. The apparatus according to the claim 13, wherein a process fluid, flow, broth or suspension is moving forwards from a beginning to an end of the process by way of rotors, screws, blows or paddlewheels.

15. The apparatus according to the claim 14, wherein the apparatus further comprising sensors or other measurement systems for measuring temperature, pH, turbidity, contents of various gases, conductivity, pO₂, pCO₂, impedance, viscosity, glucose or fructose content, or any other relevant or measurable parameter for the bioprocess, the sensors being situated at any point of the process or process flow in any of the first reactor pool and the second reactor pool.

16. The apparatus according to claim 15, wherein the process is adjusted in any of the first reactor pool and the second reactor pool with respect to one or more chosen parameters at any time point during the process flow, wherein the parameters can be temperature of 28-32° C. for a lactate-producing LAB population in the first reactor pool, and at 37-42° C. for a corresponding population in the second reactor pool.

17. The apparatus according to claim 16, wherein process control and adjustment or addition of reagents or water is being facilitated by results of measurements by the sensors.

18. The apparatus according to claim 17, wherein the mannitol is recovered by crystallization or by any other method carried out in a separate container or series of containers from fluid of the second reactor pool.

19. The apparatus according to claim 18, wherein the lactate is a main product in the first reactor pool, whereas the lactate is an additional product in the second reactor pool with same equipment being used for recovery of the lactate in both cases.

20. A biorefinery system for optimizing a simultaneous or interlinked production of lactate or Small Chain Fatty Acids (SCFA's) and mannitol, said biorefinery system comprising:
a first reactor pool configured for production of lactate or SCFA's;
a second reactor pool configured for production of mannitol using rumen bacteria as biocatalysts in such a way that after recovery of the mannitol a residual process fluid is applied to the first reactor pool for further elevating both the lactate or SCFA's and mannitol levels of a biorefining as a whole; and
a recirculation line configured for transferring at least a portion of the residual process fluid exiting the second reactor pool from a second end for introduction back into a first end of the second reactor pool opposite to that of the second end;
wherein the second reactor pool is in direct communication with the first reactor pool.
